(19) United States
(12) Patent Application Publication
(10) Pub. No.: US 2010/0145393 A1
(43) Pub. Date: Jun. 10, 2010

(54) MEDICAL AND DENTAL POROUS IMPLANTS

(75) Inventors: T. Wade Fallin, Hyde Park, UT (US); Joshua A. Butters, Chandler, AZ (US); M. Mary Sinnott, Logan, UT (US); Daniel E. Gerbec, Logan, UT (US); Daniel F. Justin, Logan, UT (US); Therese M. Stewart, Wellington, FL (US)

Correspondence Address: IMDS, INC., 124 SOUTH 600 WEST, LOGAN, UT, 84321, US

(73) Assignee: MedicineLodge, Inc., Logan, UT (US)

(21) Appl. No.: 12/329,275

(22) Filed: Dec. 5, 2008

(51) Int. Cl. A61B 17/56

(52) U.S. Cl. 606/301; 606/305; 606/308; 606/331

(57) ABSTRACT

A porous PEEK implant system which may provide tissue ingrowth throughout the body of the implant. The implant may comprise a solid or porous PEEK core. The porous PEEK implants may be of general shape and size but provide the structure necessary to carry out the function of the implant. The porous PEEK implant pores may be seeded with agents to encourage tissue ingrowth as well as other agents such as antibiotics, anesthetics, analgesics and the like.

### CROSS-REFERENCE TO RELATED APPLICATIONS

This application claims the benefit of the following, which are incorporated herein by reference:

pending prior U.S. Provisional Patent Application No. 60/012304, filed 7 Dec. 2007, which carries Applicants' docket no. MLI-68 PROV, and is entitled Porous PEEK ACL Interference Screw;

pending prior U.S. Provisional Patent Application No. 60/012299, filed 7 Dec. 2007, which carries Applicants' docket no. MLI-69 PROV, and is entitled Porous PEEK suture Anchor;

pending prior U.S. Provisional Patent Application No. 60/012296, filed 7 Dec. 2007, which carries Applicants' docket no. MLI-70 PROV, and is entitled Osteochondral Bone Platform;

pending prior U.S. Provisional Patent Application No. 60/012291, filed 7 Dec. 2007, which carries Applicants' docket no. MLI-71 PROV, and is entitled Porous PEEK Implant Fixation Surface (Pegs, Posts, Etc.); and

pending prior U.S. Provisional Patent Application No. 60/012288, filed 7 Dec. 0 2007, which carries Applicants' docket no. MLI-72 PROV, and is entitled Delivery of Drug Solution to Bone-Implant Interfaces Through the Use of Porous Material.

### BACKGROUND OF THE INVENTION

1. The Field of the Invention

The present invention relates generally to implants and hardware, and more particularly, to systems requiring bony ingrowth, pathways to provide nutrients and drug diffusion.

2. The Relevant Technology

Currently, implants including anchors, screws, pegs and posts typically rely on barbs, threads, physical force and other artificial means to attach to bones and tissues and maintain their location in those bones and tissues. Still, other implants rely on a porous metal coating to allow bony ingrowth.

Current implants, such as rotator cuff anchors and other anchors used to attach soft tissue to bone, typically rely on an anchor body which, for example, may be a screw. While this connection type provides attachment it also has its drawbacks simply because current anchors often fail before healing occurs by pull-out of the anchor from the soft cancellous bone in which they are placed. Some implants have a porous metal coating to allow bony ingrowth for fixation; however, metal coatings are often too stiff and sometimes result in stress shielding, bone resorption and subsequent loosening. Some implants have a porous polymer coating on a metal implant but this is not as highly regarded because of the dissimilarity of the two materials and the difficulty with compatibly attaching the two materials.

Dental implants face similar if not the same problems, as mentioned above, often because the metal is too stiff, stress shielding and subsequent loosening.

In the spine similar problems occur with stress shielding in regards to metal intervertebral devices as well as bony through growth. Other present dilemmas include non-radiolucency and the inability to determine proliferation progress with current platforms.

Another disadvantage of traditional metal implants, with or without pores, is the inability to drill through the implant in the case of revision surgery.

As the above described implants illustrate, the existing systems for attaching bone to bone, soft tissue to bone or soft tissue to soft tissue using anchors, screws, posts, pegs and the like may not be as effective as desired and may lead to further surgeries.

### BRIEF DESCRIPTION OF THE DRAWINGS

Various embodiments of the present invention will now be discussed with reference to the appended drawings. It is appreciated that these drawings depict only typical embodiments of the invention and are therefore not to be considered limiting of its scope.

FIG. 1 illustrates a perspective side view of a porous PEEK surgical fastener;

FIG. 2 illustrates a cross-sectional side view of a porous PEEK surgical fastener;

FIG. 3 illustrates a perspective view of a porous polymer dental implant;

FIG. 3a illustrates a perspective view of a porous polymer dental implant comprising a snap fit;

FIG. 4 illustrates a perspective view of a porous PEEK intervertebral fusion cage;

FIG. 5 illustrates a perspective view of a porous PEEK mesh assembly;

FIG. 6 illustrates a perspective view of a porous PEEK bone screw;

FIG. 7 illustrates a perspective slightly side view of a porous PEEK toggle anchor assembly;

FIG. 8 illustrates a side view of a porous PEEK interlaminar scaffold between two laminae;

FIG. 9 illustrates a posterior view of a spine and a side view of a porous PEEK laminar graft scaffold;

FIG. 10 illustrates a side view two spinal process and a side view of a porous PEEK interspinous process spacer scaffold;

FIG. 11 illustrates a perspective posterior view of a facet joint and a porous PEEK interfacet scaffold;

FIG. 12 illustrates a perspective view of a porous PEEK general fusion cage.

### DETAILED DESCRIPTION

The present invention relates to systems for greater implant fixation using a polymer such as porous polyetheretherketone (PEEK) material. Those of skill in the art will recognize that the preceding description is merely illustrative of the principles of the invention, which may be applied in various ways to provide many different alternative embodiments. This description is made for the purpose of illustrating the general principles of this invention and is not meant to limit the inventive concepts in the appended claims.

One embodiment of the present invention includes a surgical fastener made from a PEEK material with pores throughout to encourage bony ingrowth throughout the implant. The fastener may be inserted into a number of locations and may include a suture anchor, a toggle anchor or other implantable anchor used in surgical procedures.

The porous PEEK implant is ideally positioned into a bone. The fastener is forced into a bone by any means known in the art for attaching a fastener to a bone. The fastener may include such agents as hydroxyapetite (HA), bone morphologic protein or bone morphogenic protein that can facilitate bony ingrowth. The bony ingrowth allows for greater fixation and implementation of the porous PEEK implant and creates a greater security that is more anatomical in nature. With the greater promotion of bony ingrowth the resistance to back-out is also greatly improved.

Ultrasonic welding of two parts comprised of the same material, for example a core of solid PEEK and an outer wall of porous PEEK, alleviates the previous concerns of material compatibility. Additionally the porous PEEK implant provides radiolucency as well as MRI compatibility to eliminate MRI scatter imaging artifacts.

A second embodiment is the use of porous PEEK in dental implants. Dental implants are one of the single most significant advancements in dentistry. Missing teeth can be replaced with stable, comfortable and natural looking and feeling artificial replacements. A porous PEEK fixation post has its advantages because the material provides better compliance and stiffness match to the surrounding bone, providing a better distribution of stresses and transferring load from the implant to the bone.

Alternate embodiments include a platform made from porous PEEK which provides the scaffold on which tissues can be grown in vitro and then implanted as a unit for repair of cartilage or other soft tissues in orthopedic joint spaces. The porous nature of the scaffold will allow for in vivo supply of nutrients to the tissue from the scaffold side. In addition, the scaffold provides support structure that has a stiffness closer to that of natural bone. Porous PEEK bone graft scaffold for use in spinal implication is equally advantageous. These advantages include that allograft bone may be difficult to obtain and may have some foreign body reaction and sterile processing concerns, and autograft bone is often painful to harvest. The scaffold would provide the mechanical structure that would function to sustain the in-vivo loading; while the autograft bone chips/morselized bone which may be distributed in the porous PEEK prior to or during implantation encourages bony fusion, ingrowth, proliferation, or overgrowth. Stress shielding may be minimized with the use of a porous PEEK implant.

Furthermore, porous PEEK implants provide a mechanical path that can continue to provide nutrients to the tissue after implantation while also providing adequate support for the tissue. These implants also afford a surgeon the opportunity to drill through the polymeric structure in case of revision surgery.

The following definitions should be used with regard to this application:. “Tissue ingrowth” means the biological growth of any tissue, including soft and bony, throughout a medical implant. “Bony ingrowth” means the biological growth of bone matter throughout a medical implant. “Solid PEEK” means the portion of the implant that is non-porous, or that there are no pores which allow tissue ingrowth. “Porous PEEK” means the portion of the implant that is porous, or that pores are large enough to allow for fluid flow and tissue ingrowth.

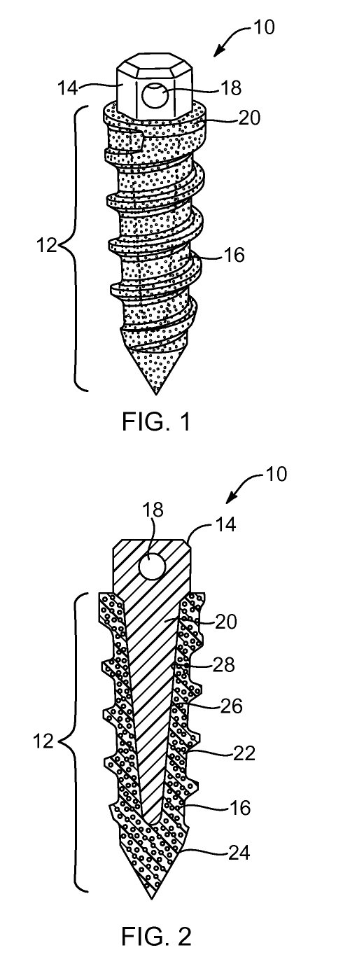

FIGS. 1 and 2 illustrate a perspective view and a cross sectional view of an embodiment of a porous PEEK surgical fastener 10 comprising a solid PEEK suture retention portion 14 at the proximal end of the surgical fastener 10 with an aperture 18 designed to receive a suture. A solid PEEK core component 20 extends longitudinally and is at least partially encompassed by a porous PEEK outer component 22 that may be ultrasonically welded in place at a complimentary interface between a core component interface surface 28 and a second component interface surface 26. At the interface, a transition portion may be formed where the solid PEEK is intermixed with the porous PEEK. The porous PEEK outer component 22 may interface with a bone as well at the tissue interface surface 24, with individual pores in communication with the tissue interface surface. A porous PEEK fastener body 12 extends longitudinally from the distal end of the suture retention portion 14. The solid PEEK core component 20 may be a continuation of the solid PEEK suture retention portion 14. The fastener body 12 may be threaded allowing for threadable insertion into a bone.

Alternatively the fastener body 12 may also be tapered to allow slidable insertion into the bone forming a type of press fit. The fastener body 12 is comprised of a core component 20, pores 16 and an outer surface 22. The pores 16 of the porous PEEK fastener body 12 provide access throughout the body 12 for bony ingrowth, with the exception of the solid PEEK core component 20. Within the porous PEEK material and the fastener body 12 and within the pores 16 may be some biological agents which promote bony ingrowth through the matrix of pores 16. These agents may be any biological compatible bone ingrowth enhancement material which may include allograft bone, autograft bone, hydroxyapatite, bone morphologic protein, bone morphogenic protein, morselized bone or any other osteobiologic material that promotes bony ingrowth.

Alternate embodiments of a surgical fastener may include a porous PEEK core (not shown) located centrally of the porous PEEK fastener body 12 that may be ultrasonically welded in place, or welded by another thermal process. The porous PEEK core may be an alteration of the solid PEEK suture retention portion 14. The porous PEEK core may allow for greater bony ingrowth throughout the fastener because of the porous core.

It will be appreciated that not only is a surgical fastener capable of this technology but any surgical fastener, suture anchor or other orthopedic anchor or screw may also be used.

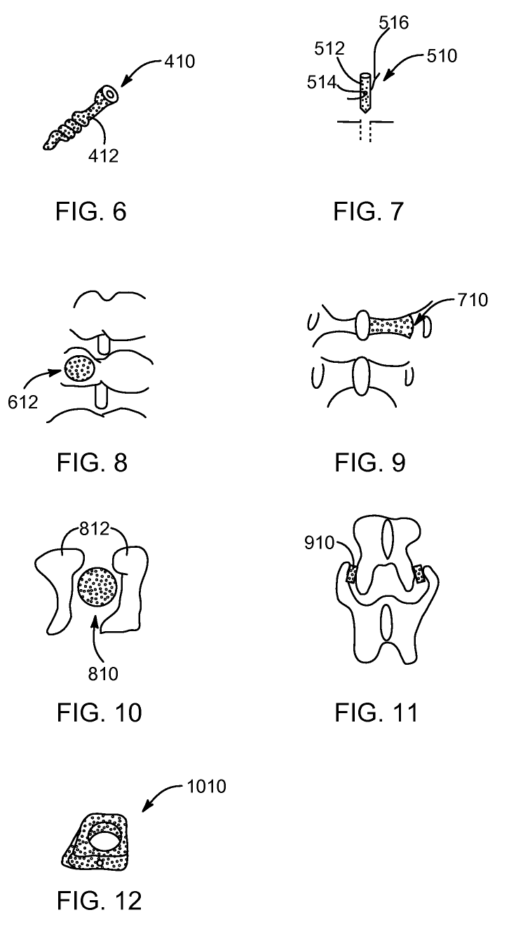

FIGS. 6 and 7 illustrate other possible embodiments of an anchor which may include a bone screw 410 which may be comprised of porous PEEK material 412 throughout the screw or may be partially comprised of porous PEEK. Another embodiment may include a porous PEEK toggle anchor 510 which may comprise pores 512. The toggle anchor 510 may comprise a toggle anchor aperture 514 in which a suture 516 passes through. After the toggle anchor 510 enters the tissue the suture 516 is pulled to move the toggle anchor 510 from a vertical position to a horizontal position fixing the toggle anchor 510 in the tissue and allowing for tissue ingrowth into the toggle anchor 510. Yet another embodiment may include a porous PEEK barrel anchor or hollow screw (not shown) similar to a toggle anchor. The barrel anchor it comprises a trough. The barrel anchor may be comprised entirely of porous PEEK and a pin or internal screw intended for insertion into the barrel anchor trough may be comprised of solid PEEK or porous PEEK similar to the toggle anchor.

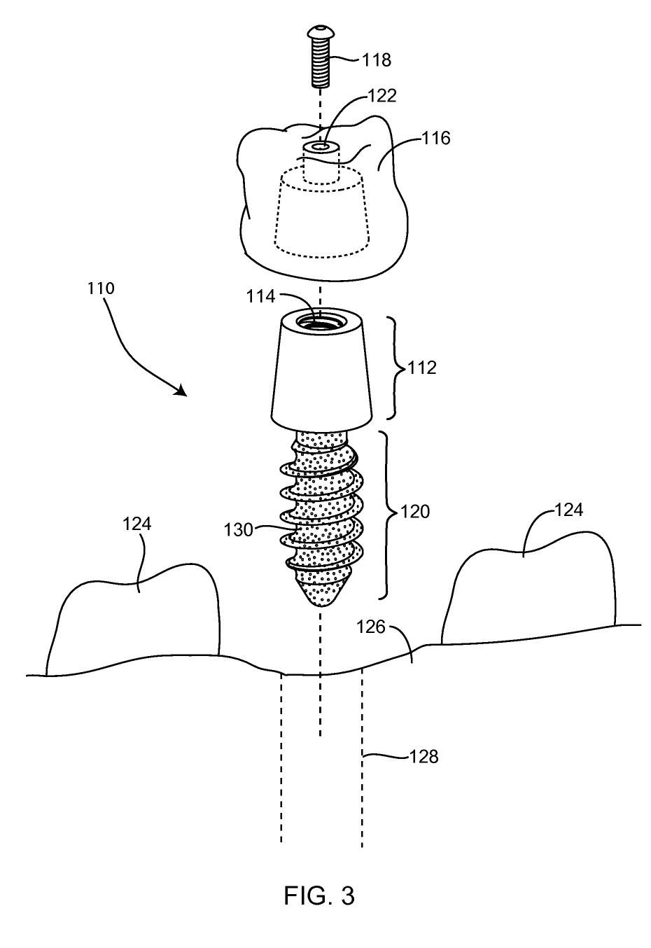

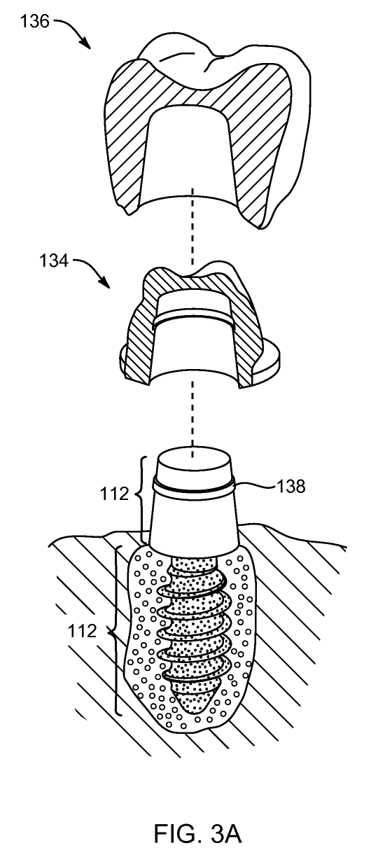

Referring to FIG. 3 a perspective view of an embodiment of a dental implant 110 comprising PEEK material. The PEEK dental implant 110 may comprise a single piece with a solid PEEK head 112 at the proximal end of the dental implant 110 and a porous PEEK fixation post 120 extending longitudinally from the distal end of the head 112. The implant 110 may be formed from a single block of PEEK which comprises solid PEEK at one end and becomes increasingly porous toward the opposite end, such that an implant monolithically formed from the block can comprise a solid PEEK head 112 and a porous PEEK post 120. Alternatively, the solid PEEK head 112 may be formed separately from the fixation post 120. The fixation post 120 also comprises an outer wall or external bone interface 132 which interfaces with the bone. Alternately, the head 112 may be a first piece ultrasonically welded to a second piece, the fixation post 120. The head 112 comprises a crown screw aperture 114 extending longitudinally through the head 112 designed to threadably or slidably receive a crown screw 118 to provide permanent fixation of a crown 116 which completely encompasses the head 12 of the dental implant 110. The crown screw 118 is threaded through a crown aperture 122 of the crown 116. The crown screw 118 engages the threads of the aperture 114. A portion of the aperture 114 may extend into the porous PEEK fixation post 120. The porous PEEK fixation post 120 comprises pores 130 through which bony or soft tissue ingrowth may take place.

The PEEK implant 110 may be fastened between teeth 124 and secured in an opening 128 created by the surgeon. The opening 128 may be threaded to engage with the external bone interface 132, which may also be threaded. The porous PEEK fixation post 120 may be threadably inserted into the opening 128 created by the surgeon in the proper place in the gum line 126, providing fixation. Alternatively the porous PEEK fixation post 120 may comprise a tapered end (not shown) and be inserted into a hole 128 created by the surgeon providing a press fit fixation. A possible third embodiment may comprise barbs (not shown) positioned on the exterior of the porous PEEK fixation post 120. The barbs would be positioned to allow one way advancement into a hole created for the dental implant 110, in addition to security and fixation of the implant.

Referring to FIG. 3a, another possible means for crown fixation may be a snap fit of a metal cast abutment 134 on the solid PEEK head 112. The snap fit may comprise either a circumferentially flange 138 on the solid PEEK head 112 or may be a circumferential groove (not shown) on the solid PEEK head 112. The metal solid cast abutment 134 provides a complimentary fit over the solid PEEK head 112 with the groove or the flange 138. A ceramic or composite crown 136 is secured to the metal solid cast abutment 134 completely encasing the cast abutment 134 within the crown 136.

The fixation post 120 may be formed entirely of porous PEEK or may be formed of a solid PEEK core and a porous PEEK outer body. While PEEK is a suitable material selection for this dental implant it will be appreciated that a variety of other polymers may be used to achieve the same function.

Alternatively the solid PEEK head 112 may be removed after the fixation post 120 is positioned in the opening 128 and the gums are then allowed to overgrow the fixation post 120 for a period of time. This time also allows for greater fixation and tissue ingrowth of the fixation post 120. The overgrowth of the gums is then cut away and the solid PEEK head 112 is reattached allowing for attachment of the cast abutment 134 and the crown 136 or attachment of the crown 116 itself.

The embodiment set forth above depicts a single tooth implant; however, it will be appreciated that this system and method may also be used for other dental implants such as multiple teeth replacement, mini implants and full denture support.

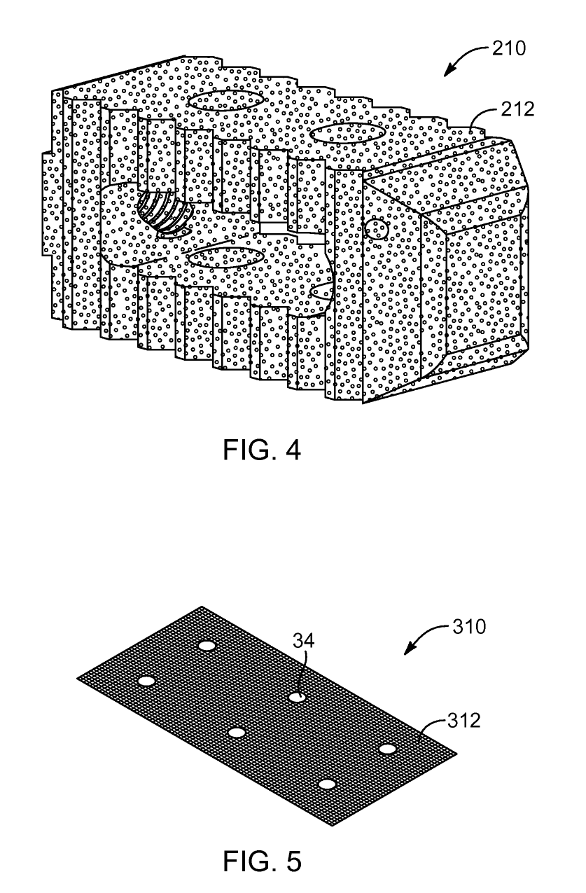

Referring to FIGS. 4-5 and 8-12, medical implants are illustrated which comprise porous PEEK material.

Referring to FIG. 4, a perspective view of a porous PEEK fusion cage 210 is shown. The general implant 210 comprises pores 212 that allow for bony ingrowth throughout the pores 212. The implant 210 need only be generally shaped for its intended purpose. For example, the fusion cage implant 210 would be comprised of porous PEEK material and would be surgically placed between two vertebrae.

Referring to FIG. 5, a porous PEEK mesh implant 310 is shown comprising pores 312 and suture apertures 314. This mesh implant 310 may be used for soft tissue repair, for example hernia repair, maintaining the general shape of repair mesh. The porous PEEK mesh implant 310 would provide for ingrowth of the tissue into the pores of the mesh and the porous PEEK provides greater security and strength than other surgical meshes.

Referring to FIG. 8, a porous PEEK interlaminar scaffold 610 is illustrated between two laminae. Once again, the porous PEEK would provide further stabilization and fusion between the two adjacent laminae.

Referring to FIG. 9, a porous PEEK laminar graft scaffold 710 is illustrated on a spine. The laminar graft scaffold 710 would provide greater protection of the dura/cord after partial, hemi, or full laminectomy, allowing for bony ingrowth into the laminar graft scaffold 710.

Referring to FIG. 10, a porous PEEK interspinous process spacer scaffold 810 is illustrated between two interspinous processes 812. The porous PEEK would allow ingrowth of bone to encourage bone fusion between spinous processes. The device may be shaped like a dowel or an “H” with flanges extending along one or more sides of the spinous processes.

Referring to FIG. 11, a porous PEEK interfacet scaffold 910 is illustrated between a facet joint of a spine. Porous PEEK material would provide for bony ingrowth of the facets into the scaffold 910 for use in slight distraction and fusion of the facets.

Referring to FIG. 12, a porous PEEK fusion cage 1010 is depicted to show an alternate embodiment of a possible fusion cage whether it be lateral, anterior or cervical. Other possible embodiments (not shown) may also include, but are not limited to, a ring for an artificial meniscus, a plug for condyle repair, or a larger structure to replace the tibial plate and/or condyles in the knee.

It will be appreciated that these illustrations are not intended to limit the scope of the invention but rather to provide examples of uses of the porous PEEK implant. Each implant may be used for its intended structure and purpose. It will be appreciated further that while PEEK is one suitable material selection, the preceding implants may also be formed of other polymers known in the art, including, but not limited to, polyamides such as poly[parphenylene terphthalamide], polyaryletherketone or other thermoplastic polymers such as polysulfone.

The implants as described herein may also be pre-loaded or seeded with agents to promote ingrowth into the implant. As previously disclosed, for potential bony ingrowth the implant may be loaded with autograft, allograft, bone morphologic and morphogenic proteins and HA, among others. Additionally, these implants may also be loaded pharmaceutical agents such as anesthetics, antimicrobials, analgesics, orthobiologics, growth proteins and growth factors, among others. The pores in the PEEK material may provide for delayed release of these agents as well. The release of these agents may also provide a method for uniform distribution throughout the bone/implant interface or soft-tissue/implant interface.

The present invention may be embodied in other specific forms without departing from its spirit or essential characteristics. For example, above are described alternative examples of inserts and implants. It is appreciated that various features of the above-described examples of each can be mixed and matched to form a variety of other combinations and alternatives. It is also appreciated that this system should not be limited to a single method of use. The implants and/or inserts system may be used for any surgery requiring implant placement or replacement. As such, the described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A surgical fastener, comprising:
a core component formed entirely of PEEK, the core component having a first component interface surface; and
an outer component coupled to the core component, the outer component formed entirely of porous PEEK, the outer component having a second component interface surface complementary in shape to the first component interface surface, the outer component further having a tissue interface surface.

2. The surgical fastener of claim 1, wherein the core component is formed entirely of non-porous PEEK.

3. The surgical fastener of claim 1, wherein the core component is formed entirely of porous PEEK.

4. The surgical fastener of claim 1, wherein the fastener is selected from the group consisting of a screw, a bone anchor, an interference screw, a suture anchor, an anchor, an anterior cruciate ligament fixation screw, a dental anchor, and a dental screw.

5. The surgical fastener of claim 1, wherein the first component interface surface and the second component interface surface are permanently joined together by a thermal process.

6. The surgical fastener of claim 5, wherein the thermal process is ultrasonic welding.

7. The surgical fastener of claim 5, further comprising a transition portion formed between the core component and the outer component, the transition portion comprising intermixed non-porous PEEK and porous PEEK.

8. The surgical fastener of claim 1, wherein the outer component comprises a plurality of pores in communication with the tissue interface surface, the surgical fastener further comprising a biologically compatible bone ingrowth enhancement material selected from the group consisting of autograft bone, allograft bone, hydroxyapatite, bone morphogenic protein, bone morphologic protein, and morselized bone; wherein the bone ingrowth enhancement material is distributed within the pores.

9. The surgical fastener of claim 1, wherein the outer component comprises a plurality of pores in communication with the tissue interface surface, the surgical fastener further comprising a therapeutic agent selected from the group consisting of orthobiologics, anesthetics, analgesics, antimicrobial agents, growth proteins, and growth factors; wherein the therapeutic agent is distributed within the pores.

10. A system comprising:
a medical implant shaped to be coupled to a first tissue, the medical implant comprising:
an implant body, the implant body comprising an first interface surface formed monolithically with the implant body, the first interface surface shaped to be attached to a portion of the first tissue;
wherein the medical implant is formed entirely of porous PEEK.

11. The system of claim 10, wherein the first tissue is a first bone.

12. The system of claim 11, wherein the implant body further comprises a second interface surface formed monolithically with the implant body, the second interface surface shaped to be attached to a portion of a second bone.

13. The system of claim 12, wherein the first bone is a first vertebra and the second bone is a second vertebra adjacent the first vertebra, wherein the implant body is selected from the group consisting of an interbody device, an interlaminar scaffold, a laminar graft, an interspinous process device, an interfacet block, an anterior cervical cage, and an interbody cage.

14. The system of claim 11, wherein the implant body comprises a plurality of pores in communication with the interface surface, the system further comprising a biologically compatible bone ingrowth enhancement material selected from the group consisting of autograft bone, allograft bone, hydroxyapatite, bone morphogenic protein, bone morphologic protein, and morselized bone; wherein the bone ingrowth enhancement material is distributed within the pores.

15. The system of claim 11, the system further comprising a surgical fastener, the surgical fastener shaped to couple the implant body to the first bone, wherein the surgical fastener urges the interface surface to remain in contact with the portion of the first bone.

16. The system of claim 10, wherein the first tissue is a soft tissue, and wherein the implant body is a mesh structure.

17. The system of claim 10, wherein the implant body comprises a plurality of pores in communication with the interface surface, the system further comprising a therapeutic agent selected from the group consisting of orthobiologics, anesthetics, analgesics, antimicrobial agents, growth proteins, growth factors, allograft material, and autograft material; wherein the therapeutic agent is distributed within the pores.

18. A dental implant system, comprising:
a post implantable in a bone, the post comprising:
a bone-contacting portion comprising a bone engagement feature, the bone-contacting portion formed entirely of a porous polymer; and
a connection portion comprising a non-porous polymer, the connection portion having a first coupling feature;
a cast abutment comprising a second coupling feature engageable with the first connection feature to provide a connection between the cast abutment and the post; and
a dental crown mountable on the cast abutment.

19. The dental implant system of claim 18, wherein the porous polymer is porous PEEK, and wherein the non-porous polymer is non-porous PEEK.

20. The dental implant system of claim 19, wherein the cast abutment is formed of metal.

21. The dental implant system of claim 19, wherein the connection is a snap connection, wherein the first coupling feature comprises a deformable flange and the second coupling feature comprises a groove shaped to receive the deformable flange.

22. The dental implant system of claim 18, wherein the bone-contacting portion comprises a plurality of pores, the system further comprising a biologically compatible bone ingrowth enhancement material selected from the group consisting of autograft bone, allograft bone, hydroxyapatite, bone morphogenic protein, bone morphologic protein, and morselized bone; wherein the bone ingrowth enhancement material is distributed within the pores.

23. A method of implanting a dental prosthetic in an oral cavity, the method comprising:
implanting a bone-contacting portion of a polymer post into one of a mandible or a maxilla, the bone-contacting portion formed entirely of a porous polymer, the polymer post further comprising a connection portion, the connection portion comprising a non-porous polymer; and
securing the dental prosthetic to the connection portion of the polymer post.

24. The method of claim 23, wherein the porous polymer is porous PEEK, and wherein the non-porous polymer is non-porous PEEK.

25. The method of claim 23, wherein the dental prosthetic is secured to the connection portion of the polymer post prior to implanting the bone-contacting portion of the polymer post into the mandible or maxilla.

26. The method of claim 23, wherein the dental prosthetic is secured to the connection portion of the polymer post after implanting the bone-contacting portion of the polymer post into the mandible or maxilla.

27. The method of claim 23, wherein the connection portion comprises a polymer flange, wherein the dental prosthetic comprises a metal groove shaped to receive the flange, wherein securing the dental prosthetic to the connection portion comprises snapping the metal groove onto the polymer flange.
